(19) United States
(12) Patent Application Publication
(10) Pub. No.: US 2007/0160671 A1
(43) Pub. Date: Jul. 12, 2007

(54) Biguanide formulations

(75) Inventors: Xiu-Xiu Cheng, Weston, FL (US); Chih-Ming Chen, Taipei (TW); Steve Jan, Coral Springs, FL (US); Dacheng Tian, Miramar, FL (US)

Correspondence Address: DAVIDSON, DAVIDSON & KAPPEL, LLC, 485 SEVENTH AVENUE, 14TH FLOOR, NEW YORK, NY, 10018, US

(73) Assignee: Andrx Corporation, Weston, FL (US)

(21) Appl. No.: 11/724,931

(22) Filed: Mar. 16, 2007

Related U.S. Application Data
(63) Continuation of application No. 10/442,692, filed on May 20, 2003.
(60) Provisional application No. 60/382,651, filed on May 23, 2002.
(60) Provisional application No. 60/382,652, filed on May 23, 2002.

(51) Int. Cl. A61K 9/14; A61K 31/155

(52) U.S. Cl. 424/484; 514/635

(57) ABSTRACT

In certain embodiments, the invention is directed to a pharmaceutical dosage form consisting essentially of a single phase matrix comprising metformin or a pharmaceutically acceptable salt thereof and at least one controlled release excipient; said dosage form providing a mean Tₘₐₓ of metformin from about 3 to about 12 hours after administration to human patients.

This application is a continuation of U.S. application Ser. No. 10/442,692, filed May 20, 2003, which claims priority from U.S. Provisional Application Nos. 60/382,651 and 60/382,652 both filed on May 23, 2002, the disclosures of which are hereby incorporated by reference in their entireties.

### FIELD OF THE INVENTION

The present invention relates to controlled release unit dose formulations comprising a biguanide, e.g., metformin or pharmaceutically acceptable salts thereof such as metformin hydrochloride or the metformin salts described in U.S. Pat. Nos. 3,957,853 and 4,080,472 which are incorporated herein by reference.

### BACKGROUND OF THE INVENTION

In the prior art, many techniques have been used to provide controlled and extended-release pharmaceutical dosage forms in order to maintain therapeutic levels of medicaments and to minimize the effects of missed doses of drugs caused by a lack of patient compliance.

WO 99/47128 describes a controlled release delivery system for metformin which requires a two phase system which includes an inner solid particulate phase formed of substantially uniform granules containing metformin and one or more hydrophilic polymers, one or more hydrophobic polymers and one or more hydrophobic materials, and an outer continuous phase in which the above granules are embedded and dispersed throughout. The outer continuous phase includes one or more hydrophilic polymers, one or more hydrophobic polymers and one or more hydrophobic materials.

U.S. Pat. No. 6,099,859 describes a controlled release antihyperglycemic tablet which has a semipermeable coating on a core with a passageway formed in the membrane to allow for osmotic diffusion of the drug from the core.

There exists a need in the pharmaceutical industry for a controlled release dosage form of metformin or a pharmaceutically acceptable salt thereof which provides effective control of blood glucose with once a day dosing. Such dosage form would provide a pharmacokinetic profile similar to once a day metformin dosage forms currently available (Glucophage XR) without the necessity for a two phase formulation as described in the prior art.

### OBJECTS AND SUMMARY OF THE INVENTION

It is an object of the present invention to provide a controlled release dosage form of a biguanide, e.g., metformin or a pharmaceutically acceptable salt thereof, which provides effective control of blood glucose levels in human patients.

It is a further object of the present invention to provide a method of treating human patients with non-insulin-dependent diabetes mellitus (NIDDM) on a once-a-day basis with a biguanide, e.g., metformin or a pharmaceutically acceptable salt thereof.

It is a further object of the present invention to provide formulations for treating human patients with non-insulin-dependent diabetes mellitus (NIDDM) which may be administered on a once-a-day basis wherein the formulation comprises a biguanide or a biguanide and a second antidiabetic agent, and methods thereof.

In certain embodiments, the present invention is directed to a pharmaceutical dosage form consisting essentially of a single phase matrix comprising metformin or a pharmaceutically acceptable salt thereof and at least one pharmaceutically acceptable excipient; said dosage form providing a mean Tₘₐₓ of metformin from about 3 to about 12 hours after administration to human patients.

In certain embodiments, the present invention is directed to a pharmaceutical dosage form comprising a single phase matrix comprising metformin or a pharmaceutically acceptable salt thereof and at least one pharmaceutically acceptable excipient; said dosage form providing a mean Tₘₐₓ of metformin from about 3 to about 12 hours after administration to human patients, said dosage form not having a controlled release coating.

In certain embodiments, the present invention is directed to a pharmaceutical dosage form comprising a single phase matrix comprising less than 60% metformin or a pharmaceutically acceptable salt thereof and greater than 40% of a water soluble polymer.

In certain embodiments, the present invention is directed to a pharmaceutical dosage form consisting essentially of a single phase matrix comprising less than 70% metformin or a pharmaceutically acceptable salt thereof and at least one pharmaceutically acceptable excipient.

In certain embodiments, the present invention is directed to a pharmaceutical dosage form comprising a single phase matrix comprising a granulation consisting essentially of metformin or a pharmaceutically acceptable salt thereof; and at least one pharmaceutically acceptable excipient.

In certain embodiments, the present invention is directed to a pharmaceutical dosage form comprising a single phase matrix comprising metformin or a pharmaceutically acceptable salt thereof and at least one pharmaceutically acceptable excipient, said dosage form providing an in-vitro dissolution of greater than 25% to about 40% metformin or salt thereof released after 1 hour; from about 30% to about 60% metformin or salt thereof released after 2 hours; from about 40% to about 70% metformin or salt thereof released after 3 hours; from about 50% to about 80% metformin or salt thereof released after 4 hours; from about 60% to about 90% metformin or salt thereof released after 8 hours, and from about 70% to about 99% metformin or salt thereof released after 10 hours.

In certain embodiments, the present invention is directed to a method of treating type 2 diabetes in a patient in need thereof comprising administering to said patient a dosage form as disclosed herein.

In certain embodiments, the present invention is directed to a method of preparing a pharmaceutical dosage form comprising compressing a mixture comprising metformin or a pharmaceutically acceptable salt thereof and at least one pharmaceutically acceptable excipient into a single phase matrix, wherein said dosage form consists essentially of said matrix; said dosage form providing a mean Tₘₐₓ of metformin from about 3 to about 12 hours after administration to human patients.

In certain embodiments, the present invention is directed to a method of preparing a pharmaceutical dosage form consisting essentially of compressing a mixture consisting essentially of metformin or a pharmaceutically acceptable salt thereof and at least one pharmaceutically acceptable excipient into a single phase matrix; said dosage form providing a mean Tₘₐₓ of metformin from about 3 to about 12 hours after administration to human patients.

In certain embodiments, the present invention is directed to a method of preparing a pharmaceutical dosage form comprising compressing a mixture comprising metformin or a pharmaceutically acceptable salt thereof and at least one water soluble polymer into a single phase matrix wherein said water soluble polymer comprises greater than 40% of said matrix.

In certain embodiments, the present invention is directed to a method of preparing a pharmaceutical dosage form consisting essentially of compressing a mixture comprising metformin or a pharmaceutically acceptable salt thereof and at least one water soluble polymer into a single phase matrix wherein said metformin comprises less than 70% of said matrix.

In certain embodiments, the present invention is directed to a method of preparing a pharmaceutical dosage form comprising compressing a mixture comprising a granulation consisting essentially of metformin or a pharmaceutically acceptable salt thereof and at least one pharmaceutically acceptable excipient into a single phase matrix.

In certain embodiments, the present invention is directed to a method of preparing a pharmaceutical dosage form comprising compressing a mixture comprising metformin or a pharmaceutically acceptable salt thereof and at least one pharmaceutically acceptable excipient into a single phase matrix, wherein said dosage form provides the following in-vitro dissolution profile when tested in a USP type 2 apparatus at 75 rpms in 900 ml of simulated intestinal fluid (pH 6.8 phosphate buffer) and at 37° C:

greater than 25% to about 40% metformin or salt thereof released after 1 hour;

from about 30% to about 60% metformin or salt thereof released after 2 hours;

from about 40% to about 70% metformin or salt thereof released after 3 hours;

from about 50% to about 80% metformin or salt thereof released after 4 hours;

from about 60% to about 90% metformin or salt thereof released after 8 hours, and

from about 70% to about 99% metformin or salt thereof released after 10 hours.

The term “single phase matrix” means a matrix which is formed with the metformin being combined with excipient in only one phase, e..g, in either (i) a dry blend or (ii) a wet granulation. This definition encompasses metformin combined with excipient in a dry. granulation, followed by a wet granulation with further excipient, as the metformin is combined with all of the excipients by wet granulation (excluding lubricants). This definition also encompasses a wet granulation of metformin alone without excipient, which is subsequently dry blended or wet granulated with excipient, as the drug itself is being combined with excipient in one phase, as the initial wet granulation of the metformin is without excipient (for purposes of this definition, water is not considered an excipient in the wet granulation of metformin alone). This definition does not encompass metformin combined with excipient in a wet granulation, with the resultant granulation combined with further excipient (excluding lubricants) in a dry blend or dry granulation (which would result in a biphasic matrix).

The term “patients” means healthy patients or patients with type 2 diabetes. This term does not encompass renally impaired patients.

The term “metformin” as it is used herein means metformin base or any pharmaceutically acceptable salt e.g., metformin hydrochloride and dibasic salts such as metformin fumarate and metformin succinate.

The term “dosage form” as it is used herein means at least one formulation of the present invention (e.g. the daily dose of the metformin can be divided in two formulations, which would collectively be considered one dosage form).

The term “Cₘₐₓ” is the highest plasma concentration of the drug attained within the dosing interval, i.e., about 24 hours.

The term “Tₘₐₓ” is the time period which elapses after administration of the dosage form at which the plasma concentration of the drug attains the highest plasma concentration of drug attained within the dosing interval (i.e., about 24 hours).

The term “AUC” as used herein, means area under the plasma concentration-time curve, as calculated by the trapezoidal rule over the complete 24-hour interval.

The term “single dose” means that the human patient has received a single dose of the drug formulation and the drug plasma concentration has not achieved steady state.

The term “multiple dose” means that the human patient has received at least two doses of the drug fonnulation in accordance with the dosing interval for that formulation (e.g., on a once-a-day basis). Patients who have received multiple doses of the controlled release formulations of the invention may or may not have attained steady state drug plasma levels, as the term multiple dose is defined herein.

The term “mean”, when preceding a pharmacokinetic value (e.g. mean Tₘₐₓ) represents the arithmetic mean value of the pharmacokinetic value taken from a population of patients unless otherwise specified (e.g. geometric mean).

### DETAILED DESCRIPTION

The formulations of the present invention are meant to encompass formulations with metformin base, as well as any pharmaceutically acceptable salt of metformin, the preferred form being metformin hydrochloride.

The dosage forms of the present invention, when administered on a once a day basis, can contain, e.g., from about 500 mg to about 2500 mg of metformin. As discussed, such daily dose may be contained in one dosage form of the invention, or may be contained in more than one such dosage form. For example, a controlled-release metformin dosage form may be formulated to contain about 1000 mg of metformin, and two of the dosage form may be administered together to provide once-a-day metformin therapy. The daily dose of the metformin may range, e.g., from about 500 mg to about 2500 mg, from about 1000 mg to about 2500 mg, or from about 2000 mg to about 2500 mg, depending on the clinical needs of the patient.

In certain embodiments, the controlled-release metformin dosage forms of the present invention can provide a mean Cₘₐₓ of metformin from about 500 to about 700 ng/ml with 500 mg metformin hydrochloride included therein. In certain embodiments, the controlled-release metformin dosage forms of the present invention provide a mean Cₘₐₓ of metformin of about 600 ng/ml with 500 mg metformin hydrochloride included therein.

In certain embodiments, the controlled-release metformin dosage forms of the present invention can provide a mean Tₘₐₓ at from about 3 to about 12 hours after oral administration. In certain embodiments, the controlled-release metformin dosage forms of the present invention provide a mean Tₘₐₓ at from about 4 to about 8 hours after oral administration.

In certain embodiments, the controlled-release metformin dosage forms of the present invention can provide a mean AUC₀₋₄₈ of metformin from about 3,500 to about 7,500 ng·hr/ml with 500 mg metformin hydrochloride included therein. In certain embodiments, the controlled-release metformin dosage forms of the present invention provide a mean AUC₀₋₄₈ of metformin in the fasted state from about 3,500 to about 6,000 ng·hr/ml with 500 mg metformin hydrochloride included therein. In certain embodiments, the controlled-release metformin dosage forms of the present invention provide a mean AUC₀₋₄₈ of metformin in the fed state from about 5,000 to about 7,500 ng·hr/ml with 500 mg metformin hydrochloride included therein.

In certain embodiments of the invention, the once daily administration of the metformin can provide a mean AUC₀₋₂₄ₕᵣ from about 80% to about 120%, wherein the daily dose of the reference standard is equal to the once-a day dose of metformin administered in the controlled release oral dosage form of the present invention.

In alternate embodiments, the dosage form of the present invention can include a further anti-diabetic agent in addition to the biguanide. For example, the dosage form can include a sulfonylurea such as glipizide, glyburide (glibenclamide), chloropropamide, tolbutamide, acetohexamide and tolazamide, troglitazone, rosiglitazone pioglitazone darglitazone, acarbose, miglitol, or pharmaceutically acceptable salts thereof.

The controlled release excipient used in the present invention is preferably a polymer which provides a controlled release of the metformin from the single phase matrix.

The polymer can include but is not limited to a hydroxyalkylcellulose (e.g., hydroxypropyl cellulose, hydroxy propyl methyl cellulose ); polyalkylene oxide having a weight average molecular weight of 100,000 to 6,000,000 (e.g., poly(ethylene) oxide, poly(methylene oxide), poly(butylene oxide), and poly(hexylene oxide); poly(hydroxy alkyl methacrylate) having a molecular weight of from 25,000 to 5,000,000; poly(vinyl)alcohol, having a low acetal residue, which is cross-linked with glyoxal, formaldehyde or glutaraldehyde and having a degree of polymerization of from 200 to 30,000; a mixture of methyl cellulose, cross-linked agar and carboxymethyl cellulose; a hydrogel forming copolymer produced by forming a dispersion of a finely divided copolymer of maleic anhydride with styrene, ethylene, propylene, butylene or isobutylene cross-linked with from 0.001 to 0.5 moles of saturated cross-linking agent per mole of maleic anyhydride in the copolymer; Carbopol® acidic carboxy polymers having a molecular weight of 450,000 to 4,000,000; Cyanamer® polyacrylamides; cross-linked water swellable indenemaleic anhydride polymers; Goodrite® polyacrylic acid having a molecular weight of 80,000to 200,000; starch graft copolymers; Aqua-Keeps® acrylate polymer polysaccharides composed of condensed glucose units such as diester cross-linked polyglucan and the like. Other polymers which form hydrogels are described in U.S. Pat. No. 3,865,108; U.S. Pat. No. 4,002,173 and U.S. Pat. No. 4,207,893 all of which are incorporated by reference. Mixtures of the aforementioned pharmaceutically acceptable polymers may also be used.

In certain embodiments, the pharmaceutically acceptable polymer can be a water insoluble polymer including, but not limited to ethylcellulose, cellulose acetate, cellulose propionate, cellulose acetate propionate, cellulose acetate butyrate, cellulose acetate phthalate, cellulose triacetate, polymethyl methacrylate, polyethyl methacrylate, polybutyl methacrylate, polyisobutyl methacrylate, polyhexyl methacrylate, poly isodecyl methacrylate, polylauryl methacrylate, polyphenyl methacrylate, polymethyl acrylate, polyisopropyl acrylate, polyisobutyl acrylate, polyoctadecyl acrylate, polyethylene, polypropylene, polyethylene oxide, polyethylene terephthalate, polyvinyl isobutyl ether, polyvinyl acetate, polyvinyl chloride, polyurethane or a mixture thereof.

In preferred embodiments of the present invention the controlled release excipient can be a suitable water-soluble polymer, including, but not limited to polyvinylpyrrolidone (“PVP”), hydroxypropylmethylcellulose (“HPMC”), polyethyleneglycol (“PEG”), hydroxypropylcellulose, sodiumcarboxymethylcellulose, carboxymethylcellulose calcium, ammonium alginate, sodium alginate, potassium alginate, calcium alginate, propyleneglycol alginate, alginic acid, polyvinylalcohol, carbomer, potassium pectate, potassium pectinate and mixtures thereof. When the polymer is PVP, it is preferred that the PVP has an average molecular weight from about 2000 to about 3 million, and more preferably from about 7,000 to about 1,500,000. Most preferred PVP has an average molecular weight of about 40,000 (such as Povidone K30) or about 1,500,000 (such as Povidone K90). Both Povidone K30 and K90 are commercially available from BASF, Midland, Mich. When the polymer is PEG, it is preferred that the PEG has an average molecular weight from about 1,000 to about 20,000. When the polymer is HPMC, high viscosity grades of HMPC, such as commercially available Methocel K100M (Dow Chemical Co.) is preferred.

Other suitable water soluble polymers can include, but are not limited to, hydroxypropylcellulose, methyl cellulose, carboxymethyl cellulose, sodium carboxymethyl cellulose, cellulose acetate phthalate, cellulose acetate butyrate, hydroxyethyl cellulose, ethyl cellulose, polyvinyl alcohol, polypropylene, dextrans, dextrins, hydroxypropyl-beta-cyclodextrin, chitosan, copolymers of lactic and glycolic acid, lactic acid polymers, glycolic acid polymers, polyorthoesters, polyanyhydrides, polyvinyl chloride, polyvinyl acetate, ethylene vinyl acetate, lectins, carbopols, silicon elastomers, polyacrylic polymers, maltodextrins, and alpha-, beta-, and gamma-cyclodextrins, and suitable mixtures of the foregoing.

The single phase matrix of the present invention can be coated with a pharmaceutically acceptable film-coating, e.g., for stability purposes, etc. The film coat may contain a pigment and/or a barrier agent, such as hydroxypropylmethylcellulose. An example of a suitable material which may be used for such a coating is hydroxypropyl methylcellulose (e.g., Opadry®, commercially available from Colorcon, West Point, Pa.). The coating may be applied using a coating pan or a fluidized bed using an organic, aqueous or a mixture of an organic and aqueous solvent. Aqueous solvents are preferred for the overcoating procedures. The film coat preferably does not affect the release of the drug from the dosage form.

In addition to the above dosage form of the present invention can include suitable quantities of other materials, e.g. preservatives, lubricants, colorants, flavorants and glidants that are conventional in the pharmaceutical art. The quantities of these additional materials will be sufficient to provide the desired effect to the desired formulation. Specific examples of pharmaceutically acceptable carriers and excipients that may be used to formulate oral dosage forms are described in the Handbook of Pharmaceutical Excipients, American Pharmaceutical Association (1986), incorporated by reference herein.

For example, the dosage form can be admixed with suitable amounts of one or more disintegrants such as cross-linked polyvinylpyrrolidone and sodium starch glycolate (Explotab); glidants such as talc and colloidal silicon dioxide; and lubricants such as magnesium stearate and acetylated monoglycerides. These ingredients can be added to the wet granulation or the dry blend of the single phase matrix, immediately prior to tableting.

The single phase matrix of the present invention can also include an one or more inert diluent such as a pharmaceutically acceptable saccharide, including a monosaccharide, a disaccharide, a polyhydric alcohol, sodium phosphate mono-, bi-, or tri-basic and/or mixtures thereof. Examples of suitable inert pharmaceutical fillers include sucrose, dextrose, lactose, fructose, xylitol, sorbitol, mixtures thereof and the like. The preferred diluent of the present invention is lactose.

The single phase matrix of the present invention may also include a hydrophobic material. The hydrophobic polymer may be selected from an alkylcellulose such as ethylcellulose, other hydrophobic cellulosic materials, polymers or copolymers derived from acrylic or methacrylic acid esters, copolymers of acrylic and methacrylic acid esters, zein, waxes, shellac, hydrogenated vegetable oils, and any other pharmaceutically acceptable hydrophobic material known to those skilled in the art. Preferably, the hydrophobic material is ethylcellulose.

The polymers used in the present invention can be ionic or non-ionic. Examples of ionic polymers include sodium alginate, carbomer, calcium carboxymethylcellulose, sodium carboxymethylcellulose, xanthan gum, methacrylic acid-acrylic acid ethyl ester copolymer, dimethylaminoethylmethacrylate-methacrylic acid esters copolymer, cellulose acetate phthalate, hydroxymethylcellulose phthalate, hydroxymethylcellulose trimellitate and hydroxymethylcellulose maleate.

In certain embodiments, the single phase matrix can be prepared by dry granulation processes which are well known in the art. Alternatively, certain embodiments can be prepared by wet granulation processes well known in the art. In certain preferred embodiments, the single phase matrix is prepared from a mixture prepared by dry blending at least one pharmaceutically acceptable excipient with a granulation consisting essentially of metformin (preferably prepared by wet granulation).

The dosage forms prepared according to certain embodiments of the present invention preferably exhibit the following in-vitro dissolution profile when tested in a USP type 2 apparatus at 75 rpms in 900 ml of simulated intestinal fluid (pH 6.8 phosphate buffer) and at

greater than 25% to about 40% metformin or salt thereof released after 1 hour;

from about 30% to about 60% metformin or salt thereof released after 2 hours;

from about 40% to about 70% metformin or salt thereof released after 3 hours;

from about 50% to about 80% metformin or salt thereof released after 4 hours;

from about 60% to about 90% metformin or salt thereof released after 8 hours, and

from about 70% to about 99% metformin or salt thereof released after 10 hours.

When administered in-vivo, the pharmaceutical dosage form of the present invention preferably provide a Tₘₐₓ of metformin from about 3 to about 12 hours; from about 3 to about 7 hours; from about 4 to about 8 hours; from about 5 to about 12 hours; or from about 5 to about 10 hours after administration to human patients.

In preferred embodiments, the dosage forms of the present invention comprising less than 70% metformin; less than 65% metformin; or less than 55% metformin.

When the dosage form of the present invention comprises a water soluble polymer, the dosage form can comprise greater than 30%; greater than 40%; or greater than 50% water soluble polymer.

Certain embodiments of the present invention as disclosed above are compositions and methods directed to metformin or pharmaceutically acceptable salts thereof. As can be appreciated by one skilled in the art, the above embodiments are also meant to encompass compositions and methods directed to biguanides other than metformin, e.g., buformin or pharmaceutically acceptable salt thereof.

### BRIEF DESCRIPTION OF THE DRAWINGS

FIG. 1 is a graph which depicts the plasma metformin concentration vs. time profile of a single dose, n=23 biostudy of Example 10 as compared to Glucophage XR.

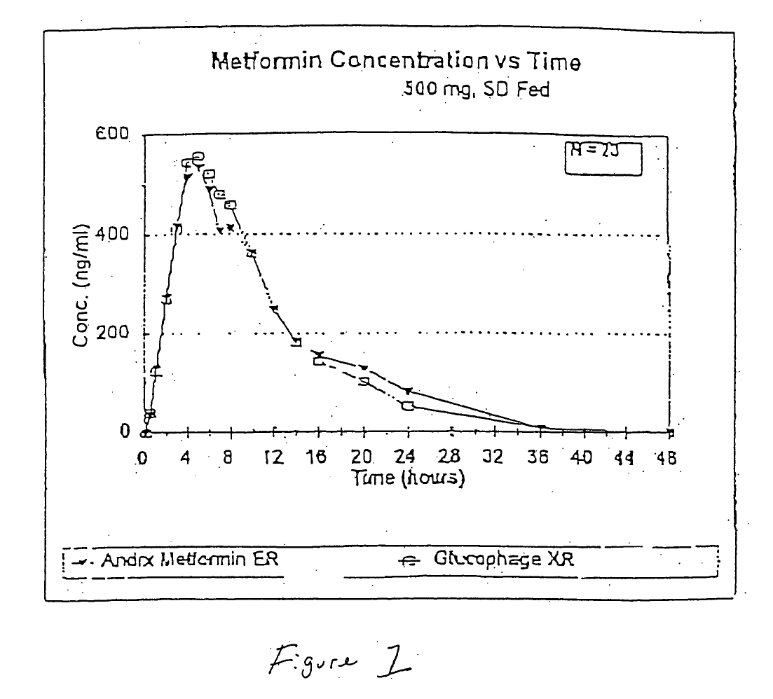

FIG. 2 is a graph which depicts the biostudy of FIG. 1, plotting log concentrations over time.

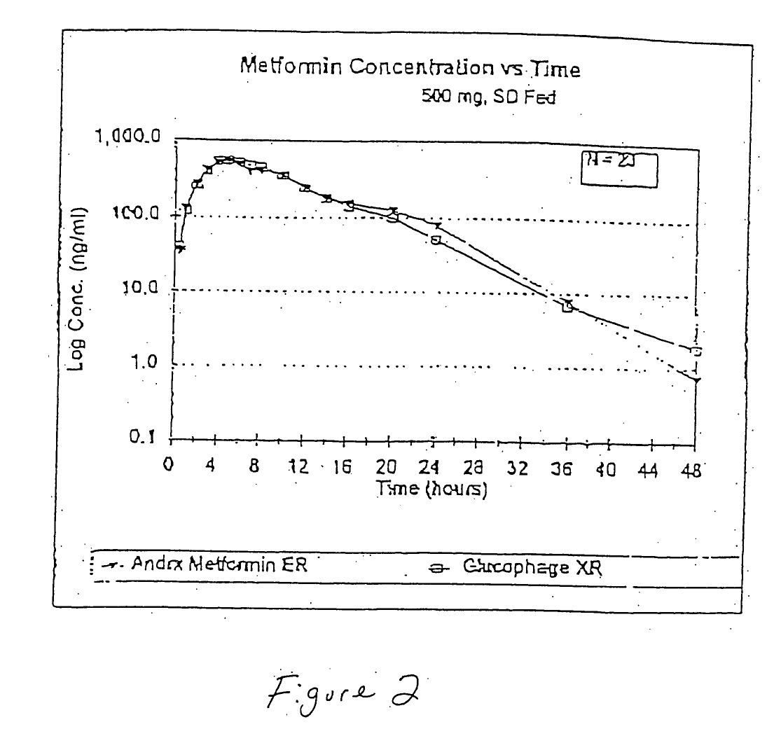

### DESCRIPTION OF CERTAIN PREFERRED EMBODIMENTS

The following examples illustrate various aspects of the present invention. They are not to be construed to limit the claims in any manner whatsoever.

### EXAMPLE 1

This formulation is prepared with the ingredients set forth in Table 1 below:

TABLE 1

| Ingredients | % Weight |
|---|---|
| Metformin HCl | 55.5 |
| Hydroxypropylmethylcellulose | 28 |
| Microcrystalline Cellulose | 15 |
| Magnesium Stearate | 1 |
| Colloidal Silicon Dioxide | .5 |
| Total | 100.00 |

The ingredients are mixed and tablets of a desired strength are made by direct compression.

### EXAMPLE 2

This formulation is prepared with the ingredients set forth in Table 2 below:

TABLE 2

| Ingredients | % Weight |
|---|---|
| Metformin HCl | 55 |
| Sodium Phosphate Tribasic | 10 |
| Hydroxypropylmethylcellulose | 30 |
| Povidone K90 | 5 |
| Total | 100.00 |

The ingredients are wet granulated in a VG-5 prior to being incorporated into a solid dosage form.

### EXAMPLE 3

This formulation is prepared with the ingredients set forth in Table 3 below:

TABLE 3

| Ingredients | % Weight |
|---|---|
| Metformin HCl | 64 |
| Hydroxypropylmethylcellulose | 30 |
| Ethylcellulose | 5 |
| Magnesium Stearate | 1 |
| Total | 100.00 |

The ingredients are wet granulated in a VG-5 with the ethylcellulose dissolved in isopropylalcohol prior to being incorporated into a solid dosage form.

### EXAMPLE 4

This formulation is prepared with the ingredients set forth in Table 4 below:

TABLE 4

| Ingredients | % Weight |
|---|---|
| Metformin HCl | 54 |
| Hydroxypropylmethylcellulose | 30 |
| Anhydrous Lactose | 10 |
| Povidone K90 | 5 |
| Magnesium Stearate | 1 |
| Total | 100.00 |

The ingredients are wet granulated in a VG-5 with the povidone dissolved in isopropylalcohol prior to being incorporated into a solid dosage form.

### EXAMPLE 5

This formulation is prepared with the ingredients set forth in Table 5 below:

TABLE 5

| Ingredients | % Weight |
|---|---|
| Metformin HCl | 50 |
| Hydroxypropylmethylcellulose | 49 |
| Magnesium Stearate | 1 |
| Total | 100.00 |

The ingredients are wet granulated in a VG-5 with the hydroxypropylmethylcellulose dissolved in water prior to being incorporated into a solid dosage form.

### EXAMPLE 6

This formulation is prepared with the ingredients set forth in Table 6 below:

TABLE 6

| Ingredients | % Weight |
|---|---|
| Metformin HCl | 40 |
| Hydroxypropylmethylcellulose | 59 |
| Magnesium Stearate | 1 |
| Total | 100.00 |

The ingredients are wet granulated in a VG-5 with the hydroxypropylrnethylcellulose dissolved in water prior to being incorporated into a solid dosage form.

### EXAMPLE 7

This formulation is prepared with the ingredients set forth in Table 7 below:

TABLE 7

| Ingredients | % Weight |
|---|---|
| Metformin HCl | 40 |
| Hydroxypropylmethylcellulose | 58 |
| Magnesium Stearate | 1 |
| Colloidal Silicon Dioxide | 1 |
| Total | 100.00 |

The ingredients are mixed and tablets of a desired strength are made by direct compression.

### EXAMPLE 8

This formulation is prepared with the ingredients set forth in Table 8 below:

TABLE 8

| Ingredients | % Weight |
|---|---|
| Metformin HCl | 49.5 |
| Hydroxypropylmethylcellulose | 44 |
| Anhydrous Lactose | 5 |
| Magnesium Stearate | 1 |
| Colloidal Silicon Dioxide | 0.5 |
| Total | 100.00 |

The metformin is wet granulated alone, to form granules consisting of the active agent. The remaining ingredients are mixed with the metformin granules and the mixture is tableted into solid dosage forms of a desired strength.

### EXAMPLE 9

This formulation is prepared with the ingredients set forth in Table 9 below:

TABLE 9

| Ingredients | % Weight |
|---|---|
| Metformin HCl | 49.5 |
| Hydroxypropylmethylcellulose | 44 |
| Anhydrous Lactose | 5 |
| Magnesium Stearate | 1 |
| Colloidal Silicon Dioxide | 0.5 |
| Total | 100.00 |

The ingredients are mixed and tablets of a desired strength are made by direct compression.

### EXAMPLE 10

This formulation is prepared with the ingredients set forth in Table 10 below:

TABLE 10

| Ingredients | % Weight |
|---|---|
| Metformin HCl | 47.5 |
| Hydroxypropylmethylcellulose | 51 |
| Magnesium Stearate | 1 |
| Colloidal Silicon Dioxide | 0.5 |
| Total | 100.00 |

The ingredients are mixed and tablets of a desired strength are made by direct compression.

### EXAMPLE 11

In-vitro dissolution testing was conducted on dosage forms prepared in accordance with Examples 7, 8, 9 and 10 in a USP type 2 apparatus at 75 rpms in 900 ml of simulated intestinal fluid (pH 6.8 phosphate buffer) and at 37° C: The results are set forth in Table 11 below:

TABLE 11

| Time | Example 7 | Example 8 | Example 9 | Example 10 |
|---|---|---|---|---|
| 0.5 | 19 | 23 | 22 | 20 |
| 1 | 27 | 33 | 32 | 29 |
| 2 | 39 | 48 | 47 | 43 |
| 3 | 48 | 59 | 58 | 53 |
| 4 | 56 | 68 | 67 | 61 |
| 6 | 68 | 81 | 81 | 74 |
| 8 | 78 | 91 | 91 | 84 |
| 10 | 85 | 97 | 97 | 90 |

### EXAMPLE 12

A 500 mg dosage form made in accordance with Examples 7 and 8 was compared to Glucophage XR in an n=9, single dose biostudy. The results are depicted in Tables 12A (fasting data) 12 B (fed data) below:

TABLE 12A

| Parameter | Glucophage ®XR | Example 7 | Example 8 |
|---|---|---|---|
| Cₘₐₓ | 674.33 | 616.33 | 808.33 |
| AUC₍₀₋ₜ₎ | 5047.77 | 4434.63 | 5359.41 |
| Tₘₐₓ | 3.56 | 3.56 | 3.56 |

TABLE 12B

| Parameter | Glucophage ®XR | Example 7 | Example 8 |
|---|---|---|---|
| Cₘₐₓ | 658.78 | 519.11 | 632.78 |
| AUC₍₀₋ₜ₎ | 6926.28 | 7162.91 | 6454.56 |
| Tₘₐₓ | 4.89 | 5.11 | 4.78 |

### EXAMPLE 13

A 500 mg dosage form made in accordance with Example 10 was compared to Glucophage XR in an n=23, single dose, fed state biostudy. The results are depicted in Table 13A (In-transformed data) and 13B (non-transformed data) below:

TABLE 13A
(Geometric Mean)

| Parameter | Glucophage ®XR | Example 10 | % Ratio |
|---|---|---|---|
| Cₘₐₓ | 584.12 | 553.58 | 94.8 |
| AUC₍₀₋ₜ₎ | 6148.93 | 6165.79 | 100.0 |
| AUC_{(0-inf)} | 6392.94 | 6536.28 | 102.0 |
| Tₘₐₓ | 5.07 | 5.59 | |

TABLE 13B
(Least Squares Mean)

| Parameter | Glucophage ®XR | Example 10 | % Ratio |
|---|---|---|---|
| Cₘₐₓ | 600.7765 | 562.5076 | 93.6 |
| AUC₍₀₋ₜ₎ | 6326.208 | 6406.783 | 101.0 |
| AUC_{(0-inf)} | 6563.599 | 6799.325 | 104.0 |

The results of this non-fasting study indicate bioequivalence between the test and the reference products

### EXAMPLE 14

A 500 mg dosage form made in accordance with Example 10 was compared to Glucophage XR in an n=28, single dose, fasted state biostudy. The results are depicted in Table 14 (In-transformed data) below:

TABLE 14
(Geometric Mean)

| | | | 90% Con. | |
|---|---|---|---|---|
| Parameter | Glucophage ®XR | Example 10 | Interval | G-Mean |
| Cₘₐₓ | 598.69 | 608.89 | (93.9; 110) | 1.020 |
| AUC₍₀₋ₜ₎ | 3949.23 | 3873.41 | (91; 106) | 0.981 |
| AUC_{(0-inf)} | 4249.18 | 4072.89 | (88.9; 103) | 0.959 |
| T_{max (hr)} | 3.55 | 3.44 | | |

The results of this fasting study indicate that the test product is bioequivalent to the reference product.

While certain preferred and alternative embodiments of the invention have been set forth for purposes of disclosing the invention, modifications to the disclosed embodiments may occur to those who are skilled in the art. Accordingly, the present invention is intended to cover all embodiments of the invention and modifications thereof which do not depart from the spirit and scope of the invention.

1. A pharmaceutical dosage form consisting essentially of:
a single phase matrix comprising metformin or a pharmaceutically acceptable salt thereof and from about 28% to about 59% w/w of at least one controlled release excipient;
said dosage form providing a mean Tₘₐₓ of metformin from 3 to 12 hours after administration to human patients.

2. A pharmaceutical dosage form comprising:
a single phase matrix comprising metformin or a pharmaceutically acceptable salt thereof and from about 28% to about 59% w/w of at least one controlled release excipient;
said dosage form providing a mean Tₘₐₓ of metformin from 3 to 12 hours after administration to human patients, said dosage form not having a controlled release coating.

3-5. (canceled)

6. A pharmaceutical dosage form comprising:
a single phase matrix comprising metformin or a pharmaceutically acceptable salt thereof and from about 28% to about 59% w/w of at least one controlled release excipient,
said dosage form providing the following in-vitro dissolution profile when tested in a USP type 2 apparatus at 75 rpms in 900 ml of simulated intestinal fluid (pH 6.8 phosphate buffer) and at 37° C:
greater than 25% to about 40% metformin or salt thereof released after 1 hour;
from about 30% to about 60% metformin or salt thereof released after 2 hours;
from about 40% to about 70% metformin or salt thereof released after 3 hours;
from about 50% to about 80% metformin or salt thereof released after 4 hours;
from about 60% to about 90% metformin or salt thereof released after 8 hours,and
from about 70% to about 99% metformin or salt thereof released after 10 hours.

7. The pharmaceutical dosage form of claim 2, wherein said single phase matrix is prepared from a dry granulation.

8. The pharmaceutical dosage form of claim 2, wherein said single phase matrix is prepared from a wet granulation.

9. (canceled)

10. The pharmaceutical dosage form of claim 2, further comprising a film coat.

11. The pharmaceutical dosage form of claim 1, wherein said dosage form does not comprise a controlled release coating.

12. The pharmaceutical dosage form of claim 2, wherein said dosage form providing the following in-vitro dissolution profile when tested in a USP type 2 apparatus at 75 rpms in 900 ml of simulated intestinal fluid (pH 6.8 phosphate buffer) and at 37° C:
greater than 25% to about 40% metformin or salt thereof released after 1 hour;
from about 30% to about 60% metformin or salt thereof released after 2 hours;
from about 40% to about 70% metformin or salt thereof released after 3 hours;
from about 50% to about 80% metformin or salt thereof released after 4 hours;
from about 60% to about 90% metformin or salt thereof released after 8 hours,and
from about 70% to about 99% metformin or salt thereof released after 10 hours.

13. The pharmaceutical dosage form of claim 2, comprising an effective amount of metformin or salt thereof for making said dosage form suitable for once-a-day dosing for type 2 diabetes in human patients.

14. The pharmaceutical dosage form of claim 6, which provides a mean Tₘₐₓ of metformin from 3 to 12 hours after administration to human patients.

15. The pharmaceutical dosage form of claim 2, which provides a mean Tₘₐₓ of metformin from about 4 to about 8 hours after administration to human patients.

16. The pharmaceutical dosage form of claim 2, which provides a mean Tₘₐₓ of metformin from about 5 to about 12 hours after administration to human patients.

17. The pharmaceutical dosage form of claim 2, which provides a mean Tₘₐₓ of metformin from about 5 to about 10 hours after administration to human patients.

18. The pharmaceutical dosage form of claim 2 which provides a mean Cₘₐₓ of metformin from about 500 to about 700 ng/ml with 500 mg metformin included therein.

19. The pharmaceutical dosage form of claim 18 which provides a mean Cₘₐₓ of metformin of about 600 ng/ml.

20. The pharmaceutical dosage form of claim 2, which provides a mean AUC₀₋₄₈ of metformin from about 3,500 to about 7,500 ng·hr/ml with 500 mg metformin included therein.

21. The pharmaceutical dosage form of claim 20 which provides a mean AUC₀₋₄₈ of metformin from about 3,500 to about 6,000 ng-hr/ml in the fasted state.

22. The pharmaceutical dosage form of claim 20 which provides a mean AUC₀₋₄₈ of metformin from about 5,000 to about 7,500 ng-hr/ml in the fed state.

23. The pharmaceutical dosage form of claim 2, comprising less than 70% metformin or pharmaceutically acceptable salt thereof.

24. The pharmaceutical dosage form of claim 2, comprising less than 65% metformin or pharmaceutically acceptable salt thereof.

25-46. (canceled)
